(19) United States
(12) Patent Application Publication
(10) Pub. No.: US 2007/0167924 A1
(43) Pub. Date: Jul. 19, 2007

(54) Pre-curved guiding catheter with eccentric balloon for supplemental backup support

(75) Inventors: Thierry Benjamin, Lowell, MA (US); James Coyle, Castlegal (IE); Raymond Godaire, Auburn, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/322,152

(22) Filed: Dec. 29, 2005

(51) Int. Cl. A61M 25/00

(52) U.S. Cl. 604/284

(57) ABSTRACT

A guiding catheter includes an elongate shaft with a main lumen and a pre-shaped curve adjacent the distal end of the shaft. The pre-shaped curve is sized and shaped for positioning in a main vessel to provide intubation of a branch vessel with a distal end of the catheter. An eccentric balloon is disposed on the shaft for selective inflation against a wall of the main vessel opposite the entrance into the branch vessel to provide supplemental backup support during interventional catheterization of the branch vessel. Methods of using the guiding catheter are also disclosed.

### FIELD OF THE INVENTION

The present invention relates generally to an intraluminal guiding catheter used during interventional catheterization, and more particularly, to a guiding catheter with a selectively inflatable eccentric balloon for supplementing back-up force provided by a pre-shaped curve in a distal region of the catheter.

### BACKGROUND OF THE INVENTION

A stenosis, lesion, or narrowing of a blood vessel such as an artery may comprise a hard, calcified substance and/or a softer thrombus material. There have been numerous interventional catheterization procedures developed for the treatment of stenoses in arteries. One of the better-known procedures is percutaneous transluminal coronary angioplasty (PTCA). According to this procedure, a narrowing in a coronary artery can be expanded by positioning and inflating a dilatation balloon across the stenosis to enlarge the lumen and re-establish acceptable blood flow through the artery. Additional therapeutic procedures may include stent deployment, atherectomy, and thrombectomy, which are well known and have proven effective in the treatment of such stenotic lesions.

In cases where the lesion targeted for treatment is located distant from a convenient vascular access location, the therapeutic procedure typically starts with the introduction of a guiding catheter into the cardiovascular system from an easily reachable site, such as through the femoral artery in the groin area or other locations in the arm or neck. The guiding catheter is advanced through the arterial system until its distal end is located near the stenosis that is targeted for treatment. During PTCA, for example, the distal end of the guiding catheter is typically inserted only into the ostium, or origin of a coronary artery. A guidewire is advanced through a main lumen in the guiding catheter and positioned across the stenosis. An interventional therapy device, such as a balloon dilatation catheter, is then slid over the guidewire until the dilatation balloon is properly positioned across the stenosis. The balloon is inflated to dilate the artery. To help prevent the artery from re-closing, a physician can implant a stent inside the dilated portion of the artery. The stent is usually delivered to the artery in a compressed shape on a stent delivery catheter and is expanded by a balloon to a larger diameter for implantation against the arterial wall.

Guiding catheters typically have a pre-shaped curve that is sized and shaped for positioning in a main vessel to orient or direct the distal end of the catheter into the entrance to a branch vessel. PTCA guiding catheters, for example, have a pre-shaped curve that fits within the aortic root and/or the ascending aorta for positioning the distal end of catheter near or within the ostium of a left or right native coronary artery or a bypass graft, depending on the curve selected. Many pre-shaped guiding catheter curves are also sized and shaped to span the width of the main vessel to support branch vessel intubation from a main vessel wall location that is contralateral, or generally opposite to the ostium of the branch vessel.

At times it is difficult to advance the interventional catheter across the stenosis because the narrowing may be very tight or the vessel(s) may have significant bends to be negotiated between the ostium and the target stenosis. In such difficult cases, the guiding catheter can fail to provide sufficient structural support or “backup” as the interventional catheter is pushed distally against resistance. In failing to provide backup support, the guiding catheter reacts to the attempted crossing forces by deforming the pre-shaped curve such that the catheter tip “backs out,” proximally from its initial position at the ostium of the branch artery. When the guiding catheter distal end remains in a fixed position, it facilitates the ability to advance the interventional catheter. As guiding catheters have advantageously evolved to have thinner walls and smaller outside diameters, it has been increasingly challenging to provide the necessary “backup support” in all clinical cases.

Catheter systems that may be utilized to increase the backup support of a conventional guiding catheter are known. In some examples, guiding catheters have one or more wire loops or leg members that may be extended against a vessel wall for bracing against forces tending to back the catheter tip out of a contralateral branch vessel. However, such wire loops may focus bracing forces in a small area, possibly embedding the loops into the vessel wall or otherwise causing injury. Disposing such wire elements near the distal end of a guiding catheter also can hinder the formation of desired curve shapes during manufacturing of the guiding catheter.

Another known guiding catheter having increased backup support includes a balloon disposed around the guiding catheter distal end that may be inflated within the ostium of a coronary artery to temporarily anchor or lock the catheter tip in place. However, inflating a balloon within the ostium of a vessel undesirably occludes blood flow into the vessel for the duration of the inflation.

There is a need to selectively reinforce the position of the distal end of a guiding catheter in its position in the ostium of a branch vessel, so that an interventional catheter can be housed therein and advanced distally into the branch vessel or a tributary vessel thereto without losing structural support or backup from the guiding catheter. The guiding catheter should have a supplemental back-up support system having minimal chance of injury to the vascular system. It is desirable for the supplemental back-up support system to be operable without occlusion of the main vessel or the intubated side branch. Other desirable features and characteristics of the present invention will become apparent from the subsequent detailed description and the appended claims taken in conjunction with the accompanying drawings.

### BRIEF SUMMARY OF THE INVENTION

The invention provides a guiding catheter including an elongate shaft with a main lumen and a pre-shaped curve adjacent the distal end of the shaft. The pre-shaped curve is sized and shaped for positioning in a main vessel to provide intubation of a branch vessel with a distal end of the catheter. An eccentric balloon is disposed on the shaft for selective inflation against a wall of the main vessel generally opposite the entrance into the branch vessel to provide supplemental backup support during interventional catheterization of the branch vessel. The eccentric balloon inflates away from the catheter shaft generally in one direction and does not inflate sufficiently to span, and thus occlude the main vessel.

A method is disclosed for using the inventive guiding catheter with selectively inflatable eccentric balloon. The method includes providing a guiding catheter having the embodiment described above; inserting the guiding catheter into a main vessel of a patient such that the catheter distal end intubates a side branch off of the main vessel; and inflating the balloon against a main vessel wall portion contralateral to the side branch vessel to reinforce the intubation of the shaft distal end in the side branch without occluding fluid flow through the main vessel.

In other embodiments of the invention, the method may also include: inserting a therapeutic device through the main lumen of the guiding catheter; positioning the therapeutic portion of the therapeutic device across the stenosis; and treating the stenosis with the therapeutic device.

### BRIEF DESCRIPTION OF THE DRAWINGS

The following drawings are illustrative of particular embodiments of the invention and therefore do not limit its scope. They are presented to assist in providing a proper understanding of the invention. The drawings are not to scale and are intended for use in conjunction with the explanations in the following detailed descriptions. Like reference numerals denote like elements in the drawings, wherein;

FIG. 1 illustrates a guiding catheter in accordance with the invention, shown deployed in the cardiovascular system of a patient;

FIG. 2 is a side view of the guiding catheter shown in FIG. 1;

FIG. 3 is a transverse cross-sectional view of the guiding catheter shown in FIG. 2, taken along line 3-3

FIG. 4 illustrates another guiding catheter in accordance with the invention, shown deployed in the cardiovascular system of a patient;

FIG. 5 is a transverse cross-sectional view of the guiding catheter and cardio-vascular system shown in FIG. 4, taken along line 5-5;

FIG. 6 is a side view of the guiding catheter shown in FIG. 4; and

FIGS. 7 and 8 illustrate a method of treating a patient, in accordance with the invention.

### DETAILED DESCRIPTION OF THE INVENTION

Specific embodiments of the present invention are now described with reference to the figures. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other passageways where it is deemed useful to selectively provide backup support for a pre-curved guiding catheter. Furthermore, there is no intention to be bound by any expressed or implied theory presented in the preceding technical field, background, brief summary or the following detailed description.

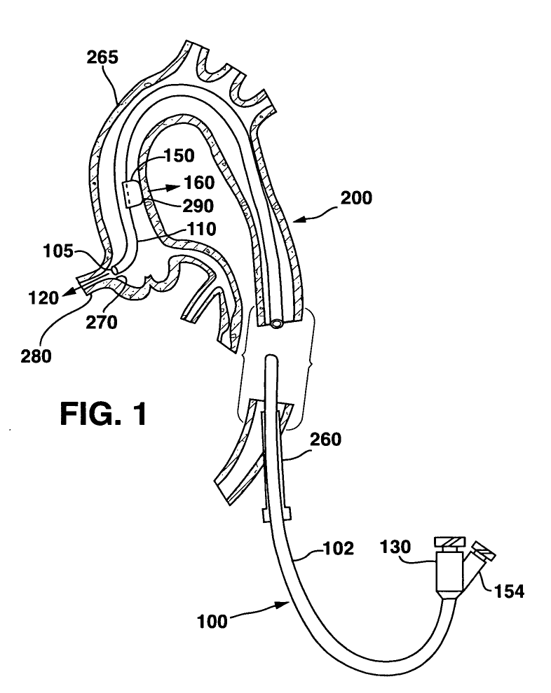

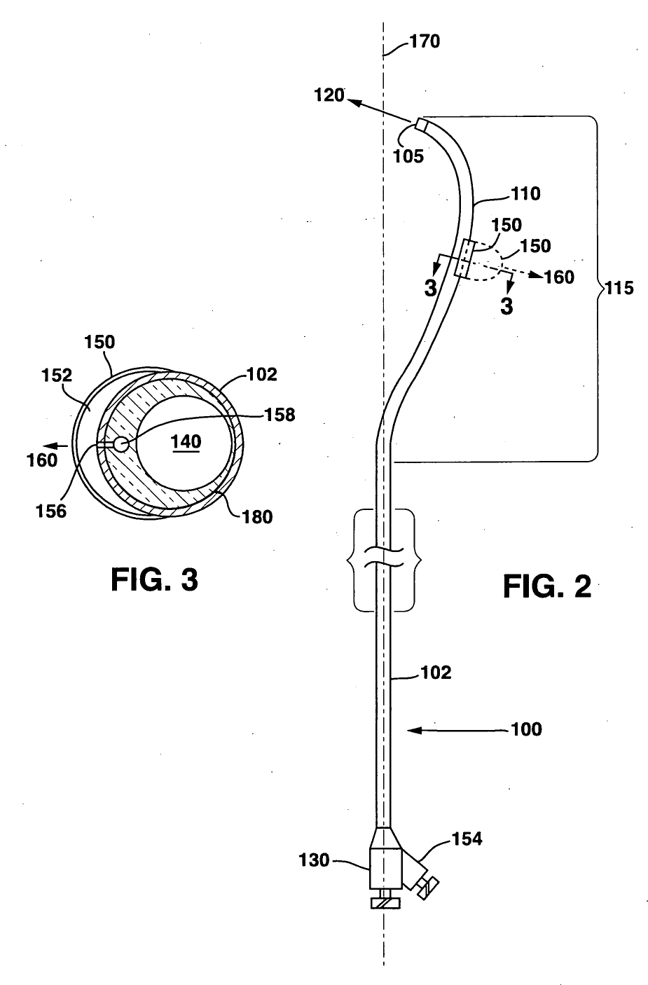

FIG. 1 illustrates guiding catheter 100 positioned within patient's vascular system 200. Catheter 100 enters vascular system 200 through introducer sheath 260, typically through a femoral artery in the groin area. Catheter 100 extends superiorly through the descending aorta, over the aortic arch 265, and inferiorly into the ascending aorta, where open distal end 105 is disposed in or near ostium 270 of right coronary artery 280. FIG. 2 illustrates pre-shaped curve 110 formed in distal region 115 of catheter shaft 102. In this example of the invention, pre-shaped curve 110 is known as a modified Judkins-type right coronary curve, named after the curve's designer, Melvin P. Judkins M.D. As shown in FIG. 1, catheter shaft 102 is sufficiently flexible such that distal region 115 may be bent around aortic arch 265. With catheter 100 positioned as shown, open distal end 105 is oriented in first direction 120, which is coaxially aligned with ostium 270 of right coronary artery 280. Guiding catheter 100 thus provides a tubular pathway for an interventional catheter (not shown) to be inserted into connector fitting 130 and passed through main lumen 140 to exit distal end 105 into coronary artery 280.

As described above, if the interventional catheter encounters high resistance when pushed into coronary artery 280, then the guiding catheter may react by distal end 105 “backing out” from its initial position at ostium 270 and deforming pre-shaped curve 110. To prevent distal end 105 from backing out of ostium 270, pre-shaped curve 110 spans the aorta from ostium 270 to the opposite or contralateral aortic wall. Pre-shaped curve 110 would typically contact the aortic wall opposite ostium 270 along buttress region 290, which is located somewhat superior to the level of ostium 270.

In accordance with the invention, the backup support provided by pre-shaped curve 110 can be selectively supplemented by inflating eccentric balloon 150 away from shaft 102 in second direction 160. As eccentric balloon 150 is inflated against buttress region 290, catheter shaft 102 is forced away from buttress region 290, as shown in FIG. 1. In this example, the supplemental backup support is provided using the principle of a third class lever wherein the force or effort provided by eccentric balloon 150 is located between the resistance or load at distal end 105 and the “fulcrum” portion of catheter shaft 102 located in the aortic arch. By inflating eccentric balloon 150, distal end 105 may be pushed back into its original position or into a deeper intubation position at ostium 270. Alternatively, if eccentric balloon 150 is inflated while distal end 105 is in its original position at ostium 270, then catheter shaft 102 will bend or deform to elastically increase backup force at distal end 105 by virtue of the axial or three-point beam bending that occurs between the “fulcrum” and load points. In this example, second direction 160 is generally parallel, offset and opposite first direction 120 both when catheter curve 110 is in the patient's body (in-vivo) and when catheter curve 110 is outside of the patient's body.

FIGS. 2 and 3 illustrate guiding catheter 100 of in FIG. 1 as it appears outside of a patient before or after use. Elongate catheter shaft 102 has longitudinal axis 170, and may comprise an optional soft tip. Pre-shaped curve 110 extends off of axis 170 such that open distal end 105 is oriented in first direction 120 sideways or lateral to axis 170. Main lumen 140 extends through shaft 102 between an open proximal end at connector fitting 130 and distal end 105. Main lumen 140 has a low-friction surface and is sized and shaped to receive and direct there through a variety of treatment devices such as guidewires and/or therapeutic devices including, but not limited to balloon catheters and stent delivery systems.

Balloon 150 is mounted eccentrically to shaft 102 and is shown in FIG. 2 in the collapsed or deflated configuration clinging to shaft 102. Broken lines in FIG. 2 also illustrate an alternate view of balloon 150 in the inflated configuration expanded away from shaft 102 in second direction 160. Balloon interior 152 is fluidly connected to inflation fitting 154 at the proximal end of catheter 100 via port 156 and inflation lumen 158. As shown in FIG. 3, inflation lumen 158 may be a passageway extending through shaft 102 alongside main lumen 140. Alternatively, the catheter shaft can be a coaxial assembly (not shown) with the inflation lumen formed as an elongate annular passageway surrounding main lumen 140.

Balloon 150 may be a patch of thin elastic material having its perimeter bonded eccentrically to shaft 102 around port 156. Balloon 150 may be formed from an elastic material such as latex, silicone elastomer, or other viscous forms of natural and synthetic rubbers such as butadiene/acrylonitride copolymers, copolyesters, ethylene vinylacetate (EVA) polymers, ethylene/acrylic copolymers, ethylene/propylene copolymers, polyalkylacrylate polymers, polybutadiene, polybutylene, polyethylene, polyisobutylene, polyisoprene, polyurethane, styrenebutadiene copolymers, and styrene-ethylene/butylene-styrene. Alternative embodiments of eccentric balloon 150 are possible, including a short tubular balloon mounted off-center or alongside shaft 102. Balloon 150 may also comprise a thin, flexible, foldable, inelastic material such as polyamide, polyethylene, polypropylene, polyurethane, polyesters, or polyethylene block amide copolymer, or polyethylene terephthalate. Balloon 150 may be attached to shaft 102 using any suitable manner known in the art, such as adhesive bonding or heat bonding. During use, it is preferred that eccentric balloon 150 not be sufficiently inflated to fully occlude a main vessel, such as the aorta, even if the size, shape and/or expansibility of balloon 150 would allow such inflation.

Catheter shaft 102 is a flexible tube that is designed to advance through a patient's vasculature to remote arterial locations without buckling or undesirable bending. Any one of a number of pre-shaped curves may be incorporated into guiding catheter 100, such as Judkins-type or Amplatz-type curves, as non-limiting examples. Curve 110 may be pre-shaped utilizing various known methods including, but not limited to, the method disclosed in U.S. Pat. No. 5,902,287 entitled “Guiding Catheter and Method of Making Same.”

Catheter shaft 102 may be constructed of one or more flexible biocompatible materials, including, but not limited to polyamide, polyethylene, polypropylene, polyurethane, polyesters, or polyethylene block amide copolymer. Catheter shaft 102 may also include layer 180 of braided filaments that resist kinking and enhance longitudinal transmission of rotation. To further aid in advancing guiding catheter 100 through the patient's vasculature, it may be desirable to vary the stiffness of catheter shaft 102 by varying the braid pitch, by varying the properties of materials used in construction, or by combining both techniques.

Main lumen 140 of guiding catheter 100 may provide a slippery interior surface for reducing frictional forces between the interior surface and devices that may be moved through main lumen 140. In one exemplary embodiment, the interior surface is provided with a slippery coating, such as a silicone compound or a hydrophilic polymer. In another exemplary embodiment, the interior surface includes a liner formed from a slippery material. Those with skill in the art may appreciate that any one of numerous low-friction, biocompatible materials such as, for example, fluoropolymers (e.g. PTFE, FEP), polyolefins (e.g. polypropylene, high-density polyethylene), or polyamides, may be used for main lumen 140.

As shown in FIGS. 1 and 2, connector fitting 130 is coupled to, and provides a functional access port at the proximal end of catheter shaft 102. Fitting 130 has a central opening in communication with main lumen 140 to allow passage of liquids or therapeutic devices there through. Connector fitting 130 may be made of metal or of a hard polymer, e.g. medical grade polycarbonate, polyvinyl chloride, acrylic, acrylonitrile butadiene styrene (ABS), or polyamide, that possesses the requisite structural integrity, as is well known to those of skill in the art.

Inflation fitting 154 is also coupled to the proximal end of shaft 102 and has an opening in fluid communication with inflation lumen 158. A source of inflation fluid (not shown) may be connected to inflation fitting 154 for inflating and deflating eccentric balloon 150. Suitable inflation fluids may include carbon dioxide gas or dilute radiographic contrast media. Fitting 154 may be made of the same or similar material as those mentioned above with respect to connector fitting 130, or fitting 154 may be integrally formed therewith.

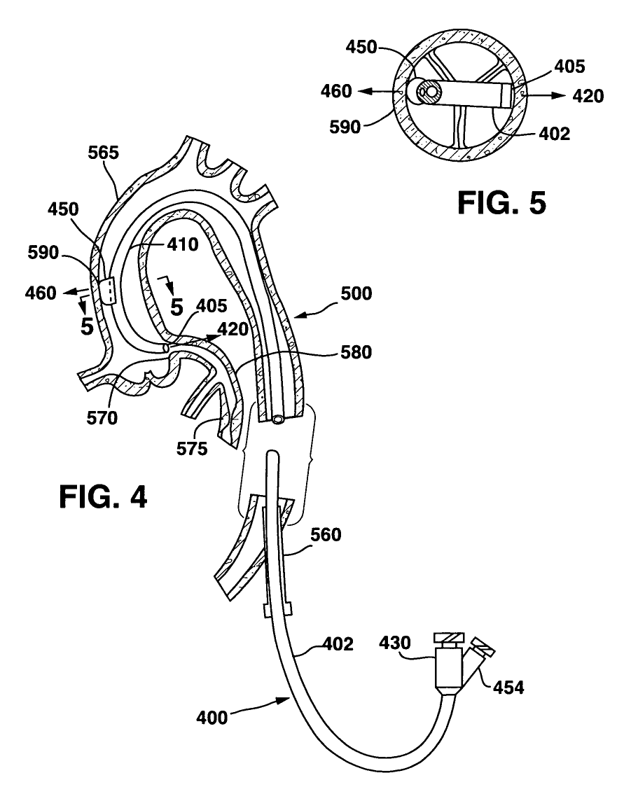

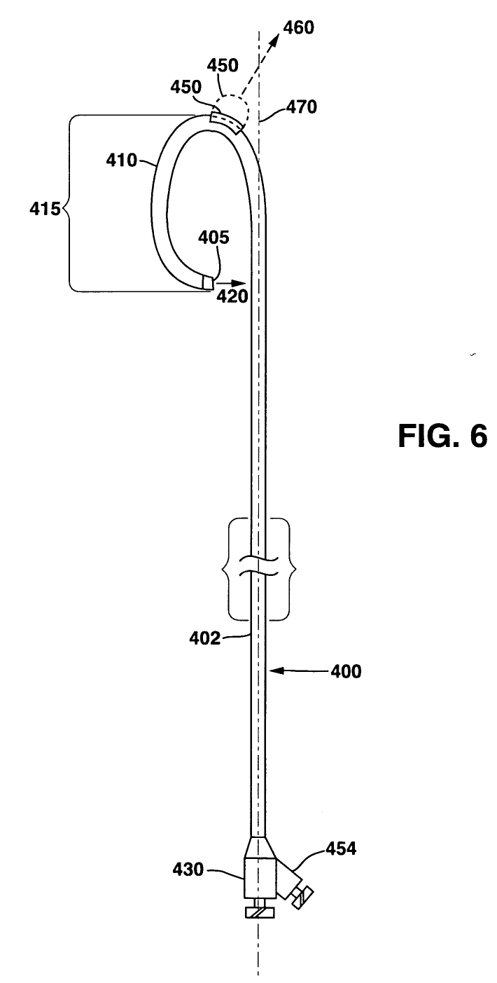

FIGS. 4-6 illustrate guiding catheter 400, which is similar to guiding catheter 100 except that catheter 400 has a different pre-shaped curve intended for intubating the left coronary artery, as shown in FIG. 4. Catheter 400 extends superiorly through the descending aorta, over aortic arch 565, and inferiorly into the ascending aorta, where open distal end 405 is disposed in or near ostium 570 of left coronary artery 580. FIG. 6 illustrates pre-shaped curve 410 formed in distal region 415 of catheter shaft 402. In this example of the invention, pre-shaped curve 410 is known as a modified Judkins-type left coronary curve. As shown in FIG. 4, catheter shaft 402 is sufficiently flexible such that distal region 415 may be bent around aortic arch 565. With catheter 400 positioned as shown, open distal end 405 is oriented in first direction 420, which is coaxially aligned with ostium 570 of left coronary artery 580. Guiding catheter 400 thus provides a tubular pathway for an interventional catheter (not shown) to be inserted into connector fitting 430 and passed through main lumen 140 to exit distal end 405 into coronary artery 580.

As described above, if the interventional catheter encounters high resistance when pushed into coronary artery 580, then the guiding catheter may react by distal end 405 “backing out” from its initial position at ostium 570 and deforming pre-shaped curve 410. To prevent distal end 405 from backing out of ostium 570, pre-shaped curve 410 spans the aorta from ostium 570 to the opposite or contralateral aortic wall. Pre-shaped curve 410 would typically contact the aortic wall opposite ostium 570 along buttress region 590, which is located somewhat superior to the level of ostium 570.

Similar to guiding catheter 100, the backup support provided by pre-shaped curve 410 can be selectively supplemented by inflating eccentric balloon 450 away from shaft 402 in second direction 460. As eccentric balloon 450 is inflated against buttress region 590, catheter shaft 402 is forced away from buttress region 590, as shown in FIGS. 4 and 5. By inflating eccentric balloon 450, distal end 405 may be pushed back into its original position or into a deeper intubation position at ostium 570.

FIG. 6 illustrates guiding catheter 400 of in FIG. 4 as it appears outside of a patient before or after use. Elongate catheter shaft 402 has longitudinal axis 470, and may comprise an optional soft tip. Pre-shaped curve 410 extends off of axis 470 such that open distal end 405 is oriented in first direction 420 sideways or lateral to axis 470. A main lumen extends through shaft 402 between an open proximal end at connector fitting 430 and distal end 405. Balloon 450 is mounted eccentrically to shaft 402 and is shown in FIG. 6 in the collapsed or deflated configuration clinging to shaft 402. Broken lines in FIG. 6 also illustrate an alternate view of balloon 450 in the inflated configuration expanded away from shaft 402 in second direction 460. Similar to guiding catheter 100, connector fitting 430 is coupled to, and provides a functional access port at the proximal end of catheter shaft 402. Fitting 430 has a central opening in communication with the main lumen to allow passage of liquids or therapeutic devices there through. Inflation fitting 454 is also coupled to the proximal end of shaft 402 and has an opening in fluid communication with an inflation lumen to inflate and deflate balloon 450.

Only when catheter curve 410 is disposed at the intended location in the patient's body, i.e. in the ascending aorta with distal end 405 located at ostium 570, then second direction 460 is generally parallel, offset and opposite first direction 420, as shown in FIGS. 4 and 5. When catheter curve 410 is outside the patient, either before or after use, then second direction 460 is not aligned with, not parallel, nor opposite to first direction 420, as shown in FIG. 6. When catheter 400 is inserted into the patient and distal region 415 is positioned in the ascending aorta, then pre-shaped curve 410 elastically opens up such that second direction 460 is generally parallel, offset and opposite first direction 420.

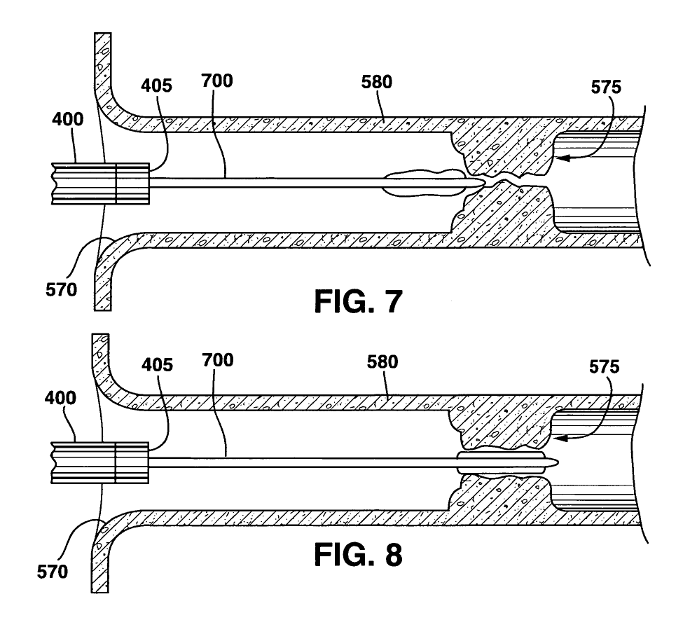

An exemplary method of using guiding catheter 400 will now be described with reference to FIGS. 4-8. The clinician confirms that balloon 450 is in the deflated or collapsed configuration and inserts the distal end of guiding catheter 400 through introducer sheath 560 into vascular system 500, typically through a femoral artery in the groin area. Guiding catheter 400 is advanced through the aorta and aortic arch 565 until catheter distal end 405 is located in ostium 570 of the targeted branch artery. FIG. 4 illustrates guiding catheter 400 positioned within patient's vascular system 500 for use with a therapeutic device. In the example shown, the branch artery is a patient's left coronary artery 580. Pre-shaped curve 410 spans the aorta from ostium 570 to the contralateral aortic wall. The portion of pre-shaped curve 410 near eccentric balloon 450 contacts the aortic wall opposite ostium 570 at a location along buttress region 590. As shown in FIG. 7, an interventional device such as PTCA catheter 700 is passed through guiding catheter 400, out of catheter distal end 405, and through left coronary artery 580 until catheter 700 reaches stenosis 575.

If attempts to push PTCA catheter 700 through stenosis 575 meet with sufficiently high resistance such that guiding catheter distal end 405 backs out of ostium 570, then the clinician may elect to inflate eccentric balloon 450 to supplement the inherent backup support already provided by pre-shaped curve 410 of guiding catheter 400. A source of balloon inflation fluid (not shown), such as a syringe and stopcock may be connected to inflation fitting 454. The clinician operates the source of fluid to inflate eccentric balloon 450 against buttress region 590 of the aorta. Varying the amount of fluid injected into the interior of balloon 450 will adjust the extent to which catheter shaft 402 is deformed away from buttress region 590 of the aorta, thus varying the amount of backup support supplemented to pre-shaped curve 410 of guiding catheter 400.

When sufficient backup support is provided by guiding catheter 400, then PTCA catheter 700 may be forced into stenosis 575 and inflated therein, as shown in FIG. 8. Before withdrawing guiding catheter 400 at the end of the procedure, eccentric balloon 450 is deflated by withdrawing inflation fluid back into the fluid source, thus collapsing balloon 450 such that it clings closely to shaft 402 for withdrawal through introducer sheath 560.

While at least one exemplary embodiment has been presented in the foregoing detailed description, it should be understood that various changes can be made in the function and arrangement of elements without departing from the scope of the invention as set forth in the appended claims and the legal equivalents thereof. For example, the invention is applicable to a large variety of pre-shaped guiding catheter curves. When the catheter is placed in the patient, an eccentric balloon can selectively provide supplemental backup support by being inflated in a direction that is generally opposite to the open catheter distal end.

What is claimed is:

1. A guiding catheter comprising:
an elongate shaft having a longitudinal axis, a main lumen connecting open proximal and distal ends, and a distal region having a pre-shaped curve extending off of the axis such that the shaft distal end is oriented in a first direction lateral to the axis;
a balloon disposed eccentrically on the shaft within or adjacent to the shaft distal region and being inflatable outwardly from the shaft in a second direction; and
an inflation lumen extending through the shaft to fluidly connect a proximally mounted inflation port with an interior of the balloon.

2. The guiding catheter of claim 1, wherein the second direction is not aligned with the first direction.

3. The guiding catheter of claim 1, wherein the second direction is generally opposite the first direction.

4. The guiding catheter of claim 3, wherein the second direction is generally parallel the first direction.

5. The guiding catheter of claim 1 wherein, the pre-shaped curve is elastically deformable into an in-vivo curve shape upon insertion of the distal region into a main vessel of a patient such that the second direction is generally opposite the first direction.

6. The guiding catheter of claim 5, wherein the second direction is generally parallel the first direction.

7. The guiding catheter of claim 1, wherein the elongate hollow shaft has a reinforcement layer.

8. The guiding catheter of claim 7, wherein the reinforcement layer comprises a tubular braid.

9. The guiding catheter of claim 1 further comprising a connector fitting mounted at the shaft proximal end in communication with the main lumen.

10. The guiding catheter of claim 1, wherein the balloon has a deflated configuration that clings to the shaft.

11. The guiding catheter of claim 1, wherein the balloon comprises an elastic material.

12. The guiding catheter of claim 1, wherein the main lumen is sized and shaped to pass an interventional device there through.

13. A guiding catheter for intubating a branch vessel off of a main vessel in a patient, the catheter comprising:
an elongate shaft having a main lumen connecting open proximal and distal ends, the shaft having a pre-shaped curve in a distal region such that, when the distal region is located in the main vessel near the branch vessel, the shaft distal end is orientable laterally towards the branch vessel;
a balloon disposed eccentrically on the shaft within or adjacent to the shaft distal region and, when the shaft distal end is oriented towards the branch vessel, the balloon is inflatable against a main vessel wall portion contralateral to the branch vessel without occluding fluid flow through the main vessel; and
an inflation lumen extending through the shaft to fluidly connect a proximally mounted inflation port with an interior of the balloon.

14. A method of using a guiding catheter comprising:
providing a guiding catheter having an elongate hollow shaft, a pre-shaped distal region and an eccentric balloon disposed within or adjacent to the shaft distal region;
inserting the guiding catheter into a main vessel of a patient such that a shaft distal end intubates a side branch vessel off of the main vessel; and
inflating the eccentric balloon against a main vessel wall portion contralateral to the branch vessel to reinforce the intubation of the shaft distal end in the side branch without occluding fluid flow through the main vessel.

15. The method of claim 14 further comprising:
passing an interventional device through the guiding catheter; and
operating the therapeutic device to perform a therapeutic procedure in the branch vessel or a tributary vessel thereto.

16. The method if claim 15, wherein the therapeutic device is an angioplasty catheter and operating the therapeutic device comprises inflating a balloon to dilate a stenosis in the branch vessel or the tributary vessel thereto.

17. The method of claim 14 further comprising:
deflating the balloon into a collapsed configuration against the shaft, and withdrawing the guiding catheter from the patient.
